(19) United States
(12) Patent Application Publication
(10) Pub. No.: US 2003/0049292 A1
(43) Pub. Date: Mar. 13, 2003

(54) Hair treatments comprising zinc salts of piroctone olamine

(75) Inventors: Angelika Turowski-Wanke, Kelkheim (DE); Waltraud Simsch, Kelkheim (DE)

Correspondence Address: CLARIANT CORPORATION, INTELLECTUAL PROPERTY DEPARTMENT, 4000 MONROE ROAD, CHARLOTTE, NC, 28205, US

(73) Assignee: Clariant GmbH

(21) Appl. No.: 10/255,786

(22) Filed: Sep. 26, 2002

Related U.S. Application Data
(63) Continuation of application No. 09/740,094, filed on Dec. 18, 2000.

(30) Foreign Application Priority Data
Dec. 18, 1999 (DE) 199 61 256

(51) Int. Cl. A61K 31/555; A61K 7/00

(52) U.S. Cl. 424/401; 514/184

(57) ABSTRACT

Cosmetic preparations comprising the zinc salt of 1-hydroxy-4-ethyl-6-(2,4,4-trimethylpentyl)-2(1H)pyridone (Zn-Octopirox).

### BACKGROUND OF THE INVENTION

The present invention relates to cosmetic preparations comprising the zinc salt of 1-hydroxy-4-methyl-6-(2,4,4-trimethylpentyl)-2(1H) pyridone (Zn-Octopirox®) as antimicrobial active ingredient.

The antimicrobial effectiveness of pyridine-N-oxide derivatives and pyridone derivatives, for example 6-[4-(4-chlorophenoxy) phenoxymethy]-1-hydroxy-4-methyl-2-pyridone, 2-aminoethanol salt, is known. WO 98 55093 claims mild cleansers comprising antimicrobially active additives such as, for example, zinc pyrithione and Octopirox (piroctone olamine).

### SUMMARY OF THE INVENTION

We have now found that the zinc salt of 1-hydroxy-4-methyl-6-(2,4,4-trimethylpentyl)-2(1H)pyridone (Zinc-Octopirox) in cosmetic compositions exhibits a broad activity spectrum toward microorganisms which attack both the skin and the mucous membrane, such as bacteria, yeasts and fungi. In the presence of the Zinc-Octopirox used according to the invention, it is possible to dispense with cytotoxic agents.

The preparations according to the invention comprise the antimicrobial active ingredient Zinc-Octopirox in the amounts by weight of from 0.05% to 10%, preferably from 0.05% to 5%, particularly preferably from 0. 1% to 1.0%.

### DESCRIPTION OF THE PREFERRED EMBODIMENTS

The zinc salt of 1-hydroxy-4-methyl-6-(2,4,4-trimethylpentyl)-2(1H)pyridone to be used in the cosmetic preparations can be prepared by common processes by reacting 1-hydroxy-4-methyl-6-(2,4,4-trimethylpentyl)-2(1H)pyridone or a salt thereof, in particular the aminoethanol salt thereof, and a zinc salt, for example ZnCl₂. The zinc salt of 1-hydroxy-4-methyl-6-(2,4,4-trimethylpentyl)-2(1H)pyridone can also be prepared “in situ” by adding an inorganic zinc salt, in particular a water-soluble zinc salt, particularly preferably ZnCl₂, to a formulation comprising the salt of 1-hydroxy-4-methyl-6-(2,4,4-trimethylpentyl)-2(,1H)pyridone, for example the Na, K, Mg, Ca salt, in particular the 2-aminoethanol salt of 1-hydroxy-4-methyl-6-(2,4,4-trimethylpentyl)-2(1H)pyridone (Octopirox) or the corresponding free acid form of this compound. Here, Octopirox and Zn salt, in particular ZnCl₂, are used in molar ratios from 1:20 to 20:1, preferably from 5:1 to 1:5, particularly preferably 1:2.5. The reaction of 1-hydroxy-4-methyl-6-(2,4,4-trimethylpentyl)-2(1H)pyridone salts, in particular of the 2-aminoethanol salt, and ZnCl₂ may be incomplete, meaning that in the cosmetic preparations, a mixture of unreacted 1-hydroxy-4-methyl-6-(2,4,4-trimethylpentyl)-2(1H)pyridone salt, in particular 2-aminoethanol salt, the zinc salt and ZnCl₂ is present. 1-Hydroxy-4-methyl-6-(2,4,4-trimethylpentyl)-2(1H)pyridone or salts thereof from inorganic and organic acids are prepared by the methods described in EP 241 918, EP 646 368, DE 17 95 270, DE 22 14 608 and DE 22 34 009.

For the use of the Zn-Octopirox according to the invention, a wide variety of cosmetic preparations are suitable, in particular shampoos, scalp creams, deodorizing body cleansing compositions, care compositions and emulsions. Examples of further preparations which are suitable according to the invention which may be mentioned are the following: hair rinses, hair treatments, hair regenerating compositions, hair tonics, blow-drying lotions, hairsprays, styling creams, styling gels, hair oils, hair pomades, hair brilliantines, shower preparations, soaps, liquid soaps, deodorant sticks, deodorant spray, emulsions. Accordingly, these are all preparations which, depending on their actual intended use, are applied to the hair, to the scalp or to the skin for a relatively short or relatively long period. Furthermore, the preparations according to the invention can be “leave on” products, for example conditioning hair creams, foot sprays or creams, antibacterial face washes or body lotions.

If the cosmetic preparations according to the invention are offered as shampoos, shower preparations or lotions, then they may be formulated as clear liquids, opaque liquids (with a pearlescent effect), in the form of a cream, gel or powder or in tablet form and as aerosols.

The washing raw materials which form the basis of these compositions may be anionic, cationic, nonionic and amphoteric in nature and also be present in combination with one another. The total amount of the surfactants used in the compositions according to the invention can be between 5 and 70% by weight, preferably between 10 and 40% by weight, particularly preferably between 12 and 35% by weight, based on the finished composition.

Examples of anionic detersive substances of this type which may be mentioned are: C₁₀—C₂₀-alkyl- and -alkylenecarboxylates, alkyl ether carboxylates, fatty alcohol sulfates, fatty alcohol ether sulfates, alkylolamide sulfates and sulfonates, fatty acid alkylolamide polyglycol ether sulfates, alkanesulfonates and hydroxyalkanesulfonates, olefinsulfonates, acyl esters of isethionates, α-sulfo fatty acid esters, alkylbenzenesulfonates, alkylphenol glycol ether sulfonates, sulfosuccinates, sulfosuccinic monoesters and diesters, fatty alcohol ether phosphates, protein-fatty acid condensation products, alkyl monoglyceride sulfates and sulfonates, alkyl glyceride ether sulfonates, fatty acid methyltaurides, fatty acid sarcosinates, sulforicinoleates and acylglutamates. These compounds and mixtures thereof are used in the form of their water-soluble or water-dispersible salts, for example the sodium, potassium, magnesium, ammonium, mono-, di- and triethanolammonium and analogous alkylammonium salts.

The proportion by weight of the anionic surfactants in the compositions according to the invention is in the range from 7% to 30%, preferably 10% to 25%, particularly preferably 12% to 22%.

Suitable cationic surfactants are, for example, quaternary ammonium salts, such as di(C₁₀-C₂₄-alkyl)dimethylammonium chloride or bromide, preferably di(C₁₂-C₁₈-alkyl)dimethylammonium chloride or bromide; C₁₀-C₂₄-alkyldimethylethylammonium chloride or bromide; C₁₀-C₂₄-alkyl-trimethylammonium chloride or bromide, preferably cetyltrimethyl-ammonium chloride or bromide and C₂₀-C₂₂-alkyltrimethylammonium chloride or bromide; C₁₀-C₂₄-alkyldimethylbenzylammonium chloride or bromide, preferably C₁₂-C₁₈-alkyldimethylbenzylammonium chloride; N—(C₁₀-C₁₈-alkyl)pyridinium chloride or bromide, preferably N—(C₁₂-C₁₆-alkyl)pyridinium chloride or bromide; N—(C₁₀-C₁₈-alkyl)isoquinolinium chloride, bromide or monoalkyl sulfate; N—(C₁₂-C₁₈-alkyloylcolaminoformylmethyl)pyridinium chloride; N—(C₁₂-C₁₈-alkyl)—N-methylmorpholinium chloride, bromide or monoalkyl sulfate; N—(C₁₂-C₁₈-alkyl)—N-ethylmorpholinium chloride, bromide or monoalkyl sulfate; C₁₆-C₁₈-alkylpenta-oxethylammonium chloride; diisobutylphenoxyethoxyethyidimethylbenzylammonium chloride; salts of N,N-diethylaminoethylstearylamide and -oleylamide with hydrochloric acid, acetic acid, lactic acid, citric acid, phosphoric acid; N-acylamidoethyl-N,N-diethyl-N-methylammonium chloride, bromide or monoalkyl sulfate and N-acylaminoethyl-N,N-diethyl-N-benzylammonium chloride, bromide or monoalkyl sulfate, where acyl is preferably stearyl or oleyl. The proportion by weight of the cationic surfactants in the compositions according to the invention is in the range from 1% to 10%, preferably 2% to 7%, particularly preferably 3% to 5%.

Suitable nonionic surfactants which can be used as detersive substances are, for example: fatty alcohol ethoxylates (alkylpolyethylene glycols); alkylphenol polyethylene glycols; alkyl mercaptan polyethylene glycols; fatty amine ethoxylates (alkylaminopolyethylene glycols); fatty acid ethoxylates (acylpolyethylene glycols); polypropylene glycol ethoxylates (Pluronic); fatty acid alkylolamides (fatty acid amide polyethylene glycols); N-alkyl-, N-alkoxypolyhydroxy fatty acid amide, sucrose esters; sorbitol esters and polyglycol ethers.

The proportion by weight of nonionic surfactants in the compositions according to the invention is in the range from 1 to 20%, preferably 2 to 10%, particularly preferably 3 to 7%.

In the compositions according to the invention, it is possible to use, for example, amphoteric surfactants: N—(C₁₂-C₁₈-alkyl)-β-aminopropionates and N-(C₁₂-C₁₈-alkyl)-β-iminodipropionates as alkali metal and mono-, di- and trialkylammonium salts; N-acylamidoalkyl-N,N-dimethylacetobetaine, preferably N—(C₈-C₁₈-acyl)amidopropyl-N,N-dimethylacetobetaine; C₁₂-C₁₈-alkyldimethylsulfopropylbetaine; amphoteric surfactants based on imidazoline (commercial name: Miranol®, Steinapon®), preferably the sodium salt of 1-(β-carboxymethyloxyethyl)-1-(carboxymethyl)-2-lauryl-imidazolinium; amine oxide, e.g. C₁₂-C₁₈-alkyldimethylamine oxide, fatty acid amidoalkyldimethylamine oxide.

The proportion by weight of amphoteric surfactants in the compositions according to the invention is in the range from 0.5 to 20%, preferably 1 to 10%.

Furthermore, foam-intensifying cosurfactants from the group of alkylbetaines, alkylamidobetaines, aminopropionates, aminoglycinates, imidazoliniumbetaines and sulfobetaines, amine oxides and fatty acid alkanolamides or polyhydroxyamides can be used in the compositions according to the invention.

Preferred surfactants in the compositions according to the invention are lauryl sulfate, laureth sulfate, cocoamidopropylbetaine, sodium cocoyl glutamate, disodium laureth sulfosuccinate and coconut fatty acid diethanolamide.

The preparations according to the invention can additionally comprise further additives customary in cosmetics, such as, for example, emulsifiers, coemulsifiers, superfatting agents, fats, oils, waxes, stabilizers, biogenic active ingredients, glycerol, preservatives, pearlizing agents, dyes and fragrances, solvents, opacifiers, thickeners and dispersants, protein derivatives, such as gelatins, collagen hydrolyzates, natural and synthetic polypeptides, egg yolk, lecithin, lanolin and lanolin derivatives, fatty alcohols, silicones, silicone derivatives, for example polyquaternized polysiloxane polymers, in particular polyquaternium-4, -6, -7, -10, -15, silicone oil, dimethicones, for example diphenyldimethicones, phenyltrimethicones, polymers having three-dimensional lattice structures, deodorizing agents, substances having keratolytic and keratoplastic action, enzymes and carrier substances. Furthermore, further antimicrobial agents of a different structure can be added to the compositions according to the invention.

Examples of emulsifiers which can be used are: sorbitan esters, sorbitol esters, phosphoric esters, monoglycerides, polysorbates, polyethylene glycol mono/di-fatty acid esters, fatty acid esters with high degrees of ethoxylation, and high molecular weight silicone compounds, such as, for example, dimethylpolysiloxanes having an average molecular weight of from 10,000 to 50,000.

Suitable nonionogenic O/W coemulsifiers are addition products of 2 to 30 mol of ethylene oxide and/or 0 to 5 mol of propylene oxide to linear fatty alcohols having 8 to 22 carbon atoms, to fatty acids having 12 to 22 carbon atoms and to alkylphenols having 8 to 15 carbon atoms in the alkyl group; C₁₂-C₁₈-fatty acid mono- and diesters of addition products of 1 to 30 mol of ethylene oxide to glycerol, glycerol mono- and diesters and sorbitan mono- and diesters of saturated and unsaturated fatty acids having 6 to 22 carbon atoms and ethylene oxide addition products thereof; addition products of 15 to 60 mol of ethylene oxide to castor oil and/or hydrogenated castor oil, polyol and, in particular, polyglycerol esters, such as, for example, polyglycerol polyricinoleate and polyglycerol poly-12-hydroxystearate. Likewise suitable are mixtures of compounds from two or more of these classes of substances. The addition products of ethylene oxide and/or propylene oxide with fatty alcohols, fatty acids, alkylphenols, glycerol mono- and diesters, and sorbitan mono- and diesters or sorbitol mono- and diesters of fatty acids or to castor oil are known, commercially available products. These are homolog mixtures whose average degree of alkoxylation corresponds to the ratio of the amounts of ethylene oxide and/or propylene oxide and substrate with which the addition reaction is carried out. C₁₂-C₁₈-Fatty acid mono- and diesters of addition products of ethylene oxide to glycerol are known from DE 20 24 051 as refatting agents for cosmetic preparations.

Superfatting agents which can be used are substances such as, for example, polyethoxylated lanolin derivatives, lecithin derivatives, polyol fatty acid esters, monoglycerides and fatty acid alkanolamides, the latter also serving as foam stabilizers. Typical examples of fats are glycerides, and suitable waxes are inter alia beeswax, paraffin wax or microcrystalline waxes, optionally in combination with hydrophilic waxes, e.g. cetylstearyl alcohol.

Stabilizers which can be used are metal salts of fatty acids, such as, for example, magnesium and/or zinc stearate. The term biogenic active ingredients means, for example, plant extracts and vitamin complexes.

Suitable preservatives are, for example, phenoxyethanol, formaldehyde solution, parabens, pentanediol or sorbic acid. Suitable pearlizing agents are, for example, glycol stearic esters, such as ethylene glycol distearate, but also fatty acid monoglycol esters.

Suitable thickeners are sodium, potassium, ammonium chloride, sodium sulfate, fatty acid alkylolamides, cellulose derivatives, for example hydroxyethylcellulose, guar gum, polyvinyl alcohol, polyvinylpyrrolidone, hydroxypropyl guar gum, starch and starch derivatives, and natural gums, carboxylvinyl polymers, for example Carbopol 934, 940, 941, 956, 980, 981,1342 and 1382.

The thickeners and dispersants used are acyl derivatives and amine oxides having long-chain aliphatic groups having 8-22, preferably 14-22, particularly preferably 16-22, carbon atoms and mixtures thereof, and also long-chain ethylene glycol esters, alkanolamides, long-chain fatty acid esters, glycerol esters, alkanolamide esters and alkyldimethylamine oxides, and the dispersants mentioned in U.S. Pat. No. 4,741,855.

Particularly suitable for shampoos are ethylene glycol esters of fatty acids having 14 to 22, particularly preferably 16 to 22, carbon atoms, in particular mono- and diethylene glycol stearate. Preference is also given to stearin monoethanolamide, stearin diethanolamide, stearin isopropanolamide, stearin monoethanolamide stearate, stearyl stearate, cetyl palmitate, glyceryl stearate, stearamide diethanolamide distearate, stearamide monoethanolamide stearate, N,N-dihydrocarbyl (C₁₂-C₂₂, in particular C₁₆-C₁₈)-amidobenzoic acid and soluble salts thereof, N,N-di(C₁₆-C₁₈)-amidobenzoic acid and derivatives. The dispersants are used in concentrations of from 0.5 to 10% by weight, preferably from 0.5 to 5% by weight, particularly preferably from 1 to 4% by weight, based on the finished composition.

Suitable carrier materials are vegetable oils, natural and hydrogenated oils, waxes, fats, water, alcohols, polyols, glycerol, glycerides, liquid paraffins, liquid fatty alcohols, sterol, polyethylene glycols, cellulose and cellulose derivatives.

Antifungicidal active ingredients which may be used are ketoconazole, oxiconazole, terbinafine, bifonazole, butoconazole, cloconazole, clotrimazole, econazole, enilconazole, fenticonazole, isoconazole, miconazole, sulconazole, tioconazole, fluconazole, itraconazole, terconazole and naftifine.

In order to improve the affinity of the zinc salt of 1-hydroxy-4-methyl-6-(2,4,4-trimethylpentyl)-2(1H)pyridone used according to the invention, on the skin/scalp, it is possible to use polymers, for example cationic guar polymers in weight amounts of from 0.01 to 1.0%, preferably 0.02 to 0.25%, as described in WO 97/26854.

The antimicrobial effect of the zinc salt of 1-hydroxy-4-methyl-6-(2,4,4-trimethylpentyl)-2(1H)pyridone used according to the invention can be improved by adding polyethylene glycol (PEG), in particular having 6 to 22 EO groups, preferably PEG-12, polypropylene glycol (PPG), polyethoxy/polypropoxy copolymers, polyethyleneimines, polyethoxylated polyethyleneimines, polyethylene oxide fatty acid glycerides, ethoxylated nonylphenol, ethoxylated alcohols, polyethylene carbohydrates. Silicones, polyalkylsiloxanes, polyalkylarylsiloxanes, polyether-siloxane copolymers, as described in U.S. Pat. No. 5,104,645 and publications cited therein, improve the care action of the compositions according to the invention.

The preparations according to the invention are prepared in a manner known per se by mixing the individual components and, if necessary, further processing them in a manner appropriate for the respective type of preparation. Some of these diverse possible preparation forms are described by way of example in the working examples.

All of these preparations are also prepared—as already mentioned in the case of the shampoo—in a manner known per se, with the addition of the active ingredient used according to the invention.

The antidandruff active ingredient is incorporated into the preparations according to the invention in amounts customarily between about 0.05 and about 10%. Within this range, the concentrations of the specific preparations depend on their intended use. Certain preparation forms such as, for example, concentrates, which are to be diluted prior to use, can also have considerably higher concentrations.

For preparations which remain on the hair, such as, for example, hair lotions, hair setting compositions, creams etc., lower concentrations are used, for example of from about 0.01 to about 1% by weight, preferably 0.1 to 0.5% by weight. They are expediently used in higher concentrations for cosmetic preparations which, optionally after dilution, act on hair and scalp for only a short period, such as, for example, shampoos or hair rinses. In these cases, concentrations of from about 0.2 to about 10% by weight, preferably from about 0.5 to about 2% by weight, for example, can be expedient.

The use according to the invention of said zinc salt of 1-hydroxy-4-methyl-6-(2,4,4-trimethylpentyl)-2(1H)pyridone is distinguished in particular by numerous advantages over the prior art mentioned.

The main advantages are:

improved substantivity, resulting in low active ingredient concentrations

broad antimicrobial activity spectrum

good solubility in aqueous and nonaqueous media

### EXAMPLES

The examples below illustrate the present invention. The amounts given are based on the weight.

### Example 1

### Antidandruff Shampoo

| Component | | % by weight |
|---|---|---|
| A | Octopirox | 0.50 |
| B | Water | 10.00 |
| C | Genapol LRO liquid | 30.00 |
| D | Belsil DMC 6032 | 0.50 |
| E | Allantoin | 0.30 |
| F | Water | ad 100 |
| G | Panthenol | 1.00 |
| | Genagen CAB | 8.00 |
| | Genapol L-3 | 1.50 |
| | Dye | q.s. |
| H | NaCl | 1.50 |
| I | ZnCl₂ | 0.35 |

Preparation method:

I Stir A into B

II Stir C into I and further stir until the solution becomes clear

III Add D in portions

IV Dissolve E in F and add with stirring and gentle heating

V Stir components of G with one another

VI If required, adjust the pH

VI. Adjust the viscosity using H

VIII Stir in I

### Example 2

### Antidandruff Shampoo

| Component | | % by weight |
|---|---|---|
| A | Octopirox | 0.50 |
| B | Water | 10.00 |
| C | Genapol LRO liquid | 30.00 |
| D | Belsil DMC 6032 | 0.50 |
| E | Allantoin | 0.30 |
| | Polyquaternium 10 | 0.20 |
| F | Water | ad 100 |
| G | Panthenol | 1.00 |
| | Genagen CAB | 8.00 |
| | Genapol L-3 | 1.50 |
| | Dye | q.s. |
| H | NaCl | 1.50 |
| I | ZnCl₂ | 0.35 |

Preparation method:

I Stir A into B

II Stir C into I and further stir until the solution becomes clear

III Add D in portions

IV Dissolve E in F and add with stirring and gentle heating

V Stir components of G with one another

VI If required, adjust the pH

VII Adjust the viscosity using H

VIII Stir in I

### Example 3

### Antidandruff Shampoo

| Component | | % by weight |
|---|---|---|
| A | Octopirox | 0.50 |
| B | Water | 10.00 |
| C | Genapol LRO liquid | 30.00 |
| D | Belsil DMC 6032 | 0.50 |
| E | Allantoin | 0.30 |
| F | Water | ad 100 |
| | Merquat 550 | 0.50 |
| G | Panthenol | 1.00 |
| | Genagen CAB | 8.00 |
| | Genapol L-3 | 1.50 |
| | Dye | q.s. |
| H | NaCl | 1.50 |
| I | ZnCl₂ | 0.35 |

Preparation method:

I Stir A into B

II Stir C into I and further stir until the solution becomes clear

III Add D in portions

IV Dissolve E in F and add with stirring and gentle heating

V Stir components of G with one another

VI If required, adjust the pH

VII Adjust the viscosity using H

VIII Stir in I

### Example 4

### Antidandruff Shampoo, Intensive Shampoo

| Component | | % by weight |
|---|---|---|
| A | Octopirox | 0.75 |
| B | Water | 10.00 |
| C | Genapol LRO liquid | 35.00 |
| | Genapol SBE | 10.00 |
| D | Perfume | 0.30 |
| | Water | ad 100 |
| | Genagen CAB | 6.00 |
| | Dye | q.s. |
| E | NaCl | 1.70 |
| F | ZnCl₂ | 0.525 |

Preparation method:

I Stir A into B

II Stir C into I and further stir until the solution becomes clear

III Add D in portions

IV If required, adjust the pH

V Adjust the viscosity using E

VI Stir in I

### Example 5

### Antidandruff Shampoo With Pearlescent Effect

| Component | | % by weight |
|---|---|---|
| A | Genapol 26 L 80 | 0.50 |
| B | Dow Corning 190 | 0.20 |
| C | Octopirox | 0.40 |
| D | Water | 5.00 |
| E | Genapol LRO liquid | 35.00 |
| F | Hostapon CCG | 2.00 |
| | Perfume | 0.30 |
| | Glycerol | 1.00 |
| | Genapol TSM | 3.00 |
| | Genagen LAA | 10.00 |
| | Merquat 550 | 0.50 |
| G | Cosmedia guar | 0.10 |
| H | Water | ad 100 |
| | Citric acid | 0.30 |
| I | DOE 120 | 1.00 |
| | Preservative | q.s. |
| J | NaCl | 0.50 |
| K | ZnCl₂ | 0.28 |

Preparation method:

I Dissolve B in A with gentle warming

II Stir C with D, add E and stir until the solution is clear

III Stir in components of F into 11 one after the other

IV Stir I into III

V Dissolve G in H using Ultra Turrax and stir into IV

VI Stir I into V and stir until everything has dissolved

VII If necessary, adjust the pH (5.5-6.0)

VIII Adjust the viscosity with J

IX Stir in K

### Example 6

### Instant Vitamin Antidandruff Hair Treatment

| Component | | % by weight |
|---|---|---|
| A | Propylene glycol | 0.70 |
| B | Octopirox | 0.05 |
| C | Carbopol 980 | 0.60 |
| D | Genamin KDM-P | 0.20 |
| | Genamin CTAC | 0.50 |
| | Wheat starch | 0.50 |
| | Belsil DMC 6032 | 0.80 |
| | Dow Corning 190 | 0.50 |
| E | Water | ad 100 |
| | Panthenol | 0.20 |
| | Celquat L 200 | 0.10 |
| | Uvinol MS 40 | 0.05 |
| | Nicotinamide | 0.30 |
| | Vitamin E | 0.10 |
| | Sodium hydroxide solution | 0.80 |
| F | Cosmedia guar | 0.20 |
| G | Perfume | 0.30 |
| H | ZnCl₂ | 0.035 |

Preparation method:

I Dissolve B into A

II Melt D at about 80° C.

III Stir F into E using Ultra Turrax

IV Heat III to 80° C.

V Add C to II and directly thereafter stir in IV

VI Stir until cold

VII Stir in [lacuna] at about 35° C.

### Example 7

### Creme Shampoo

| Component | % by weight |
|---|---|
| Zn-Octopirox | 0.20 |
| Hostapon CT-Paste | 70 |
| Hostapon SCI-65 | 5 |
| Medialan LDD | 5 |
| Ethylene glycol distearate | 1.50 |
| Water | ad 100 |

Preparation method:

### Example 8

### Antidandruff Shampoo

| Component | % by weight |
|---|---|
| Zn-Octopirox | 0.30 |
| Genagen CA 050 | 5 |
| Genapol LRO, liq. | 35.0 |
| Genapol AMG | 10.0 |
| Genagen CAB | 8.0 |
| Genapol L-3 | 2.0 |
| Sodium chloride | 0.7 |
| Water | ad 100 |

Preparation method:

### Example 9

### 3 in 1 Shampoo

| Component | % by weight |
|---|---|
| Zn-Octopirox | 0.20 |
| Genapol LRO, liq. | 35.0 |
| Genapol AMG | 5.0 |
| Hostapon KCG | 5.0 |
| Hostapon SCHC | 5.0 |
| Belsin DNC 6032 | 2.0 |
| D-Panthenol | 1.0 |
| Genapol L-3 | 2.5 |
| Genagen CAB | 8.0 |
| Sodium chloride | 2.0 |
| Water | ad 100 |

Chemical names of the commercial products used

| ® Octopirox | Clariant GmbH | 1-Hydroxy-4-methyl-6-(2.4,4- |
|---|---|---|
| | | trimethylpentyl)-2(1H)pyridone, |
| | | 2-Aminoethanol salt |
| ® Genapol | Clariant GmbH | Sodium laureth sulfate |
| LRO | | |
| ® Belsin DNC | Wacker Chemie | Dimethicone copolyol acetate |
| 6032 | | |
| Allantoin | Clariant GmbH | 5-Ureidohydantoin |
| ® Genagen | Clariant GmbH | Cocoamidopropylbetaine |
| CAB | | |
| Genapol L-3 | Clariant GmbH | Fatty alcohol polyglycol ether, 3 EO |
| ® Merquart | | Polyquaternium 7 |
| 550 | | |
| Genapol SBE | Clariant GmbH | Alkylpolyglycol ether sulfosuccinate |
| Genapol 26 L | Clariant GmbH | Fatty alcohol polyglycol ether, 8 EO |
| 80 | | |
| Dow Corning | | Dimethicone copolyol |
| 190 | | |
| Hostapon KCG | Clariant GmbH | Sodium cocoyl glutamate |
| Genapol TSM | Clariant GmbH | PEG-3 distearate/sodium laureth |
| | | sulfate |
| Genagen LAA | Clariant GmbH | Sodium lauroamphoacetate |
| ® Cosmedia | | Guar hydroxypropyltrimethyl- |
| Guar | | ammonium-chloride |
| ® Gulcamat | | PEG-120 Methylglucose dioleate |
| DOE 120 | | |
| ® Carbopol | | Carbomer |
| 980 | | |
| Genamin | Clariant GmbH | Behenyltrimethylammonium chloride |
| KDM-P | | |
| Genamin | Clariant GmbH | Cetyltrimethylammonium chloride |
| CTAC | | |
| ® Celquat L | | Polyquaternium-4 |
| 200 | | |
| Uvimol MS 40 | | Benzophenone-4 |
| Vitamin E | | Tocopherol acetate |
| Hostapon CT- | Clariant GmbH | Fatty acid methyltauride sodium salt |
| Paste | | |
| Hostapon SCI- | Clariant GmbH | Fatty acid methyl isethionate sodium |
| 65 salt | | |
| ® Medialan | Clariant GmbH | Palm kernel fatty acid sarcoside |
| LD | | |
| Genagen CA | Clariant GmbH | Fatty acid monoethanolamide |
| 050 | | polyglycol ether |
| Genapol AMG | Clariant GmbH | Acylaminopolyglycol ether sulfate |
| | | magnesium salt |
| Hostapon | Clariant GmbH | Fatty acid protein condensate |
| SCHC | | |
| Belsin DNC | Wacker Chemie | Silicone oil |
| 6032 | | |

1. A cosmetic preparation comprising the zinc salt of 1-hydroxy-4-ethyl-6-(2,4,4-trimethylpentyl)-2(1H)pyridone.

2. The cosmetic preparation as claimed in claim 1, which additionally comprises the 2-aminoethanol salt of 1-hydroxy-4-methyl-6-(2,4,4-trimethylpentyl)-2(1H)pyridone and an inorganic zinc salt.

3. The cosmetic preparation as claimed in claim 1, which additionally comprises the Na or K or Mg or Ca salt of 1-hydroxy-4-methyl-6-(2,4,4-trimethylpentyl)-2(1H)pyridone and an inorganic zinc salt.

4. The cosmetic preparation as claimed in claim 1, which comprises 0.05 to 10% by weight of the zinc salt of 1-hydroxy-4-methyl-6-(2,4,4-trimethylpentyl)-2(1H)pyridone.
